(19) United States
(12) Patent Application Publication
(10) Pub. No.: US 2022/0062478 A1
(43) Pub. Date: Mar. 3, 2022

(54) ANTIMICROBIAL LIGHT SYSTEMS FOR HIGH-TOUCH SURFACES, APPARATUSES, AND EQUIPMENT

(71) Applicants: Adam E. M. Eltorai, Marlborough, MA (US); Charles W. Henry, Denver, CO (US)

(72) Inventors: Adam E. M. Eltorai, Marlborough, MA (US); Charles W. Henry, Denver, CO (US)

(21) Appl. No.: 17/464,948

(22) Filed: Sep. 2, 2021

Related U.S. Application Data
(60) Provisional application No. 63/074,385, filed on Sep. 3, 2020.

(51) Int. Cl. A61L 2/10; H05B 41/36

(52) U.S. Cl. CPC A61L 2/10; H05B 41/36

(57) ABSTRACT

A light-based sterilization source directs an antimicrobial light at a touched or contacted surface for eliminating harmful viral or bacterial contaminants. The antimicrobial light is outside the range of harmful UV light while delivering an effective decontaminating light source for eradication of pathogenic microorganisms.

An acrylic or other transparent medium conducts the antimicrobial light from a source to an irradiated target surface for continual decontamination, and may define a backlighting arrangement.

### RELATED APPLICATIONS

This patent application claims the benefit under 35 U.S.C. § 119(e) of U.S. Provisional Patent App. No. 63/074,385, filed Sep. 3, 2020, entitled “ANTIMICROBIAL LIGHT SYSTEMS FOR HIGH-TOUCH SURFACES, APPARATUSES, AND EQUIPMENT,” incorporated herein by reference in entirety.

### BACKGROUND

Physical contact represents a viable transmission path for many harmful bacterial and viral contaminants. Indirect contact through intermediate surfaces can be mitigated through frequent cleaning of surfaces prone to contact from multiple people in a short time, such as handrails, doorknobs, elevator buttons, and the like. Chemical disinfectants are one effective means to keeping commonly touched surfaces free of transmittable disease, however can be labor intensive if done with sufficient regularity. Radiation from certain light sources can also be effective, however the radiation may also be harmful to humans, and thus imposes overhead to contain radiation.

### SUMMARY

A light-based sterilization source directs a blue light at a high touch surface for eliminating harmful viral or bacterial contaminants. The blue light is outside the range of harmful UV light while delivering an effective decontaminating, antimicrobial light source for eradication of pathogenic microorganisms, including SARS CoV-2. An acrylic or other transparent medium conducts the blue light from a source to an irradiated target surface for continual decontamination, and may define a backlighting arrangement.

Transmission of pathogens has long been known to occur by touch, from person to person, and indirectly, through surfaces contaminated by a contagious person and then touched by another. Modern approaches dispose hand dispensers of sanitizing gel in public places to encourage frequent usage around common surfaces that may incur touching by multiple persons in a short time. These “high touch” surfaces, such as door knobs/handles, elevators, rotating doors panels, escalators, personal devices (e.g. phones) can be vehicles for pathogen transmission.

Configurations herein are based, in part, on the observation that high touch surfaces may be managed by regular cleaning and/or hand sterilization by users. Substantial resources are deployed for pursuing a regular cleaning cycle and ensuring timely refilling of hand dispensers of alcohol gel. Unfortunately, conventional approaches suffer from the shortcoming that they depend on diligent execution, and still allow for periods of contagion depending on how long the pathogens remain viable on the high touch surface. Accordingly, configurations herein substantially overcome the shortcomings of rigorous surface cleaning by providing an irradiating light source directed to a high touch surface for immediate sterilization and pathogen removal based on the wavelength of the light. A further advantage is the benign nature of the irradiating light in the blue spectrum (around 400-470 nm) that removes it from the harmful UV spectrum.

The disclosed approach employs an irradiation device including a light source disposed for irradiating a high touch surface for sterilization. A system for sterilization of contaminated surfaces using the irradiation device includes a blue light source having a wavelength outside a harmful spectrum such as the UV (ultraviolet) spectrum. A light conduction medium is configured to conduct light from the blue light source to a target surface for sterilization, such that the blue light source has an intensity based on a duration of exposure on the target surface and a power for achieving sterilization over the duration of exposure. Optimal antimicrobial effectiveness of the blue light source is based on at least one of light distribution, intensity, output and duration

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a usage environment including high touch surfaces appropriate for use with configurations herein;

FIGS. 2A and 2B are schematic diagrams of an irradiation device for sterilization of high touch surfaces as in FIG. 1;

FIG. 3 shows a histogram of sterilization performance of a high touch surface based on a percentage of harmful pathogens remaining;

FIG. 4 is an example of a personal device case equipped with the irradiation device of FIG. 2A;

FIG. 5 is an example of a public high touch surface depicted by an elevator button using the irradiation device;

FIG. 6 shows the irradiation device in a drying rack for fluid vessels used in medical and caretaking environments;

FIGS. 7A and 7B show Automated Teller Machine (ATM) usage of the devices of FIGS. 2A and 2B;

FIG. 8 shows the irradiation device embedded in a personal device for usage with a flashlight application of the personal device, and

FIGS. 9A-9F show the irradiation device in a plurality of use cases and contexts.

### DETAILED DESCRIPTION

Depicted below is an example of various configurations of the antimicrobial light generation device. Several views and arrangements are shown; other embodiments may be apparent to those of skill in the art by slight variations to the form factor and electrical circuit as shown.

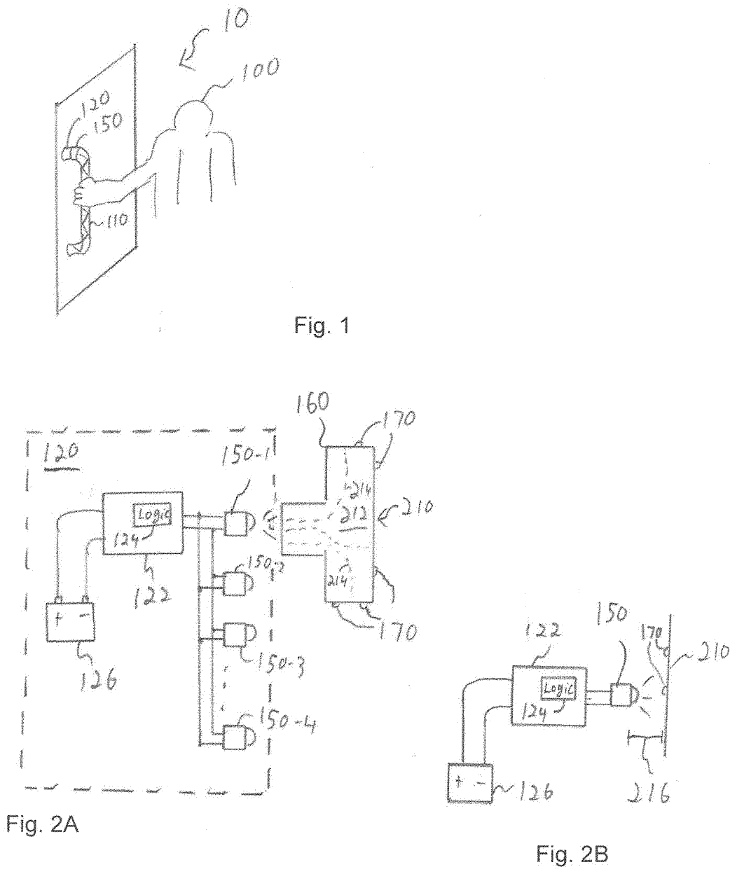

FIG. 1 is a context diagram of a usage environment 10 including high touch surfaces appropriate for use with configurations herein. Referring to FIG. 1, a human actor 100 approaches a high touch surface such as a door handle 110 formed from a translucent material. An irradiation device 120 embedded or attached at an end of the door handle includes a light source 150 disposed for irradiating the high touch surface for sterilization by reflection and refraction through the door handle material.

FIGS. 2A and 2B are schematic diagrams of an irradiation device for sterilization of high touch surfaces as in FIG. 1. Referring to FIGS. 1 and 2A-2B, the light source 150 is a blue light source having a wavelength outside a harmful spectrum. The blue light source has a wavelength between 400-470 nm, whereas ultraviolet (UV) light in conventional approaches has a wavelength around 200-300 nm. The blue light exhibits a particular effectiveness against pathogenic organisms at a wavelength substantially around 405 nm, as discussed further below. Ultraviolet (UV) light is a form of electromagnetic radiation commonly acknowledged to have a wavelength from 10 nm to 400 nm.

In FIG. 2A, the irradiation device 120 includes a controller 122 for implementing illumination logic 124, and a power supply 126 or battery to illuminate the light source 150 and for powering the controller. Power demands are minimal, such as 5 or 12 VDC, as the light source 150 may include LED (Light Emitting Diode) elements programmed or fixed at a predetermined wavelength for emitting the blue light. The light source 150 may be defined by a plurality of LED elements 150-1 . . . 150-N (150 generally), based on the power needed and the area to be irradiated.

A high touch surface 210 may be defined by any suitable surface prone for human (typically hand) contact by gripping, touching, pulling or otherwise manipulated in the normal course of usage. The high touch surface 210 may be formed from a light conduction medium 212 adapted for transporting irradiated light from the light source 150 to the high touch surface 210. In the example of FIG. 2A, reflection and refraction within the light conduction medium 212 directs the light over various paths 214 to irradiate the high touch surface from an opposed side of the touch or contact; pathogens 170 (typically small particles or droplets) receive the irradiation via the paths 214. The light source 150 is disposed appurtenant to the light conduction medium 212 such that the light conduction medium 212 includes the high touch surface 210 and is irradiated via the path 214 through the light conduction medium 212. If needed, specific mirrors may be established to provide an extended path for light conduction within the conduction medium 212.

In the example of FIG. 2A, the light conduction medium 212 includes a transparent structure 160 constructed or molded to define the high touch surface 210. Any suitable transparent or translucent shape sufficient to establish or support the light paths 214 may be formed into a high touch surface. This, of course, implies that the conduction medium 212 be devoid of filtering effects that alter the wavelength or obscurity (cloudiness) that negates the intensity, however any suitable polymer, plastic, glass or clear solid material may suffice. In generally, the transparent structure 160 is defined by a crystalline material adapted for passage of blue light, such that the crystalline material provides linear and non-linear pathways for irradiation of the light between the light source and the high touch surface. Particular compositions may include light-transmissive materials such as polymethyl methacrylate (PMMA), silica/quartz, thermoplastic polyurethane (TPU), flexible acrylic, transparent polyvinyl chloride (PVC), UV-inhibitor-free transparent PVC and solar cell material.

FIG. 2B shows an alternate arrangement where the light source 150 irradiates the high touch surface 210 from the contacted side, typically from a short distance 216 such that the intensity at the surface 210 remains of sufficient intensity for a sufficient time to sterilize or kill all or most pathogens 170. The light conduction medium therefore irradiates the target surface from a distance 216 via an atmospheric medium, in other words, is simply projected through air onto the high touch surface 210.

In contrast to conventional approaches, employing UV light around 200-300 nm, the blue light is in the visible spectrum that exhibits only nominal, non-harmful radiation. While UV light may remain an effective sterilization medium, it typically requires shielding for protection from the UV radiation. The disclosed blue light source 150 may be freely transmitted and passed through a light conduction medium such as a transparent acrylic structure appurtenant to a manual contact surface. This allows effective usage in conjunction with human activity, such as for illuminating buttons, cellphones, door handles and other applications for decontamination of surfaces prone to high contact traffic.

In specialized arrangements, the light conduction medium may be configured to refract the conducted light to the manual control surface, for passing light in a non-linear path through curved or bent acrylic structures to transparently reach the intended contact surface. This is particularly beneficial in high traffic surfaces such as handles and buttons in which the manual contact surface receives greater exposure to human epidermal regions than adjacent surfaces.

The description below illustrates that the wavelength of the light is based on an expected contamination on the target surface, and variations in exposure time and light power may be adjusted based on the time between contact occurrences with potentially contaminated fingers or hands. While SARS CoV-2 remains one of the intended contaminants for sterilization and eradication, other contaminants are also responsive to the light delivery approach as disclosed herein. The antimicrobial blue light may be at or near the 405 nm wavelength, delivered at a sufficient duration and intensity to kill the target microorganisms.

Table 1 depicts a percentage of SARS CoV-2 viral load reduction on a plastic petri dish surface post exposure to the light source 150 compared to the virus exposed to the control ambient light at each time increment.

TABLE I

| | | Triplicate samples | | | | |
|---|---|---|---|---|---|---|
| Exposure time | | # 1 | # 2 | # 3 | Mean | Std |
| 1 | min | 11.63 | 9.30 | 9.30 | 10.08 | 1.3427 |
| 2 | min | 18.85 | 16.39 | 6.56 | 13.93 | 6.5059 |
| 3 | min | 16.81 | 16.81 | 6.72 | 13.45 | 5.8220 |
| 4 | min | 35.90 | 20.51 | 28.21 | 28.21 | 7.6923 |
| 5 | min | 82.79 | 85.25 | 84.51 | 84.18 | 1.2619 |
| 10 | min | 86.70 | 85.65 | 83.83 | 85.39 | 1.4525 |
| 30 | min | 92.57 | 96.97 | 95.87 | 95.14 | 2.2917 |
| 1 | hr | 99.94 | 99.94 | 99.94 | 99.94 | 0.0038 |
| 3 | hr | 100 | 100 | 100 | 100 | 0 |

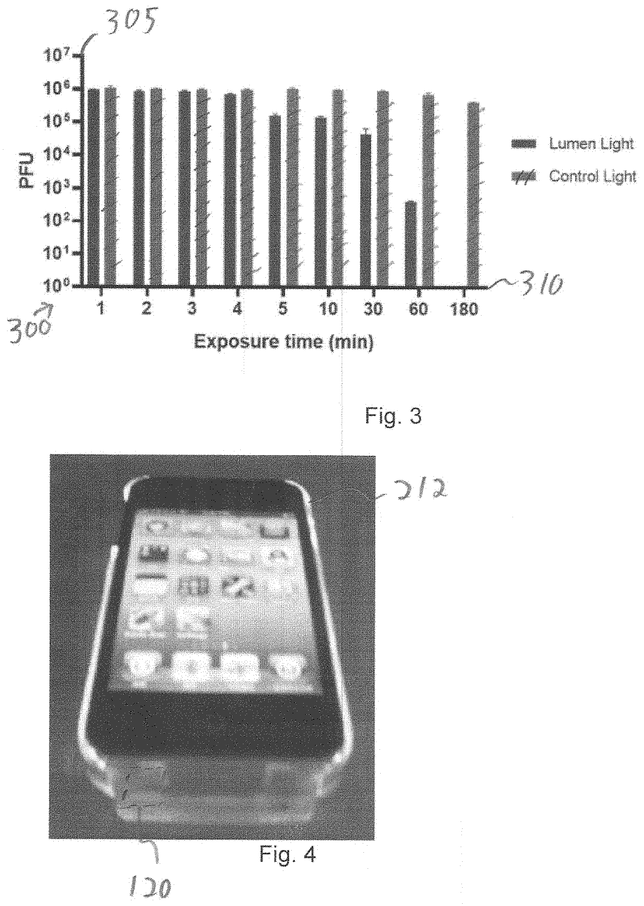

FIG. 3 shows a histogram 300 of sterilization performance of a high touch surface based on a percentage of harmful pathogens remaining. FIG. 3 evaluates the surface-killing effect of the light source 150 against the SARS CoV-2 strain, based on Table I. The light source 150 with the light facing up was installed in a biosafety cabinet with the blower on continuously at room temperature. 500 uL of virus was spread on the surface of an uncovered petri dish that was directly placed on the light source 150. A vertical axis 305 shows the plaque-forming unit (pfu) reductions from the initial populations were determined following nine continuous exposure times of 1 min, 2 min, 3 min, 4 min, 5 min, 10 min, 30 min, 1 hr or 3 hr, depicted on horizontal axis 310. At the designed time points, virus in the petri dish was recovered by adding 1 mL of MEM (Minimum Essential Medium) and then collected for plaque assay. Plaque assay was performed in duplicate and incubated for 72 hours at 37° C. 5% CO₂.

Based on FIG. 3 and Table I, it can be concluded that the viable numbers of SARS CoV-2 on the petri dish surface were significantly reduced after continuous and direct exposure to light source 150 at five minutes and longer. More than 84% viral load reduction was observed after exposing to light source 150 at five minutes and longer, compared to exposure to the control ambient light. With initial 1×106 pfu inoculum, after exposure to the light source 150 for one hour, 99.94% of viruses were eliminated; after exposure to the light source 150 for three hours, no viable virus was detected.

An advantage of the claimed approach includes the adaptability to fabricate the light source 150 and light conduction medium 212 in a variety of forms to install or retroactively apply anti-pathogen capability to any suitable high-touch surface. Various molding and formation techniques, such as injection molding, sheet fabrication or other suitable plastic or polymer based approach may be employed to form a suitably shaped light conduction medium 212 in conjunction with the light source 150 to implement an anti-pathogen, anti-microbial high touch surface. A system for sterilization of contaminated surfaces using the irradiation device 120 therefore includes an antimicrobial light source 150 having a wavelength outside a harmful spectrum, and a light conduction medium 212 configured to conduct light from the blue light source to a target surface for sterilization. The blue light source has an intensity based on a duration of exposure on the target surface and a power for achieving sterilization over the duration of exposure. Several non-limiting examples are shown in FIGS. 4-8.

FIG. 4 is an example of a personal device case equipped with the irradiation device of FIG. 2A. Referring to FIGS. 1-2B and 4, device cases are readily available for installation around personal devices (e.g. smartphones, tablets). Formation of the light conduction medium 212 in a shape for surrounding a personal device 400 extends protection to hand-held phone operation. The irradiation device 120 may be molded or attached with the light conduction medium 212 to distribute light from the blue light source 150 around the personal device 400.

The light conduction medium 212 is generally a transparent structure appurtenant to a high touch surface associated with touch based manual control. The light conduction medium 212 irradiates the target surface via refraction through the transparent structure. Alternatively, the light conduction medium irradiates the target surface from a distance via an atmospheric medium. In other words, from an external location directed towards the high touch surface but sufficiently close to achieve the expected intensity.

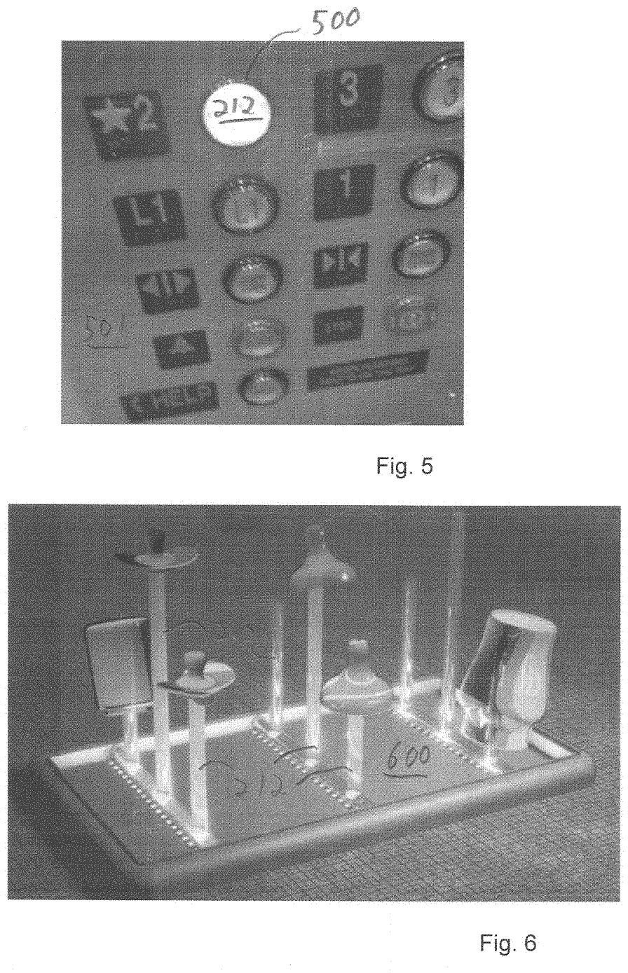

FIG. 5 is an example of a public high touch surface depicted by an elevator button using the irradiation device 120. An elevator button 500 invites successive, closely spaced touch intervals by a variety of people. Elevator buttons are often backlit from a pushbutton panel 501 disposed in the elevator. It is therefore an excellent candidate for a light conduction medium 212 formed as the translucent button and illumination instead provided by the blue light source 150.

FIG. 6 shows the irradiation device in a drying rack for fluid vessels used in medical and caretaking environments. Fluid vessels for human consumption are commonplace in a living space, and are particularly prevalent in the context of caretakers of infants, sick and elderly. Specialized vessels needing hand washing and drying invite contamination if left exposed. A drying rack 600 employs the light conduction medium 212 formed as a pillar or post for supporting inverted vessels during drying. This can be combined with a surface mounted light source 150 radiating upwards for bathing the vessels in blue light.

For either the light conduction medium 212 or external (atmospheric) radiation, illumination logic 124 further comprising control logic, such that the blue light source (light source) 150 is responsive to the control logic for identifying the target surface for sterilization, determining a material of which the target surface is formed, and computing the duration of exposure and the intensity for achieving sterilization of the target surface. The control logic may compute the duration and intensity based on a type of material defining the high touch surface. A mapping of material types to an irradiation time and intensity for the mapped surface may be employed. Generally, more porous surfaces require greater exposure to the blue light, but this may be moderated based on the material type, and also the intensity achievable with the available power supply 126.

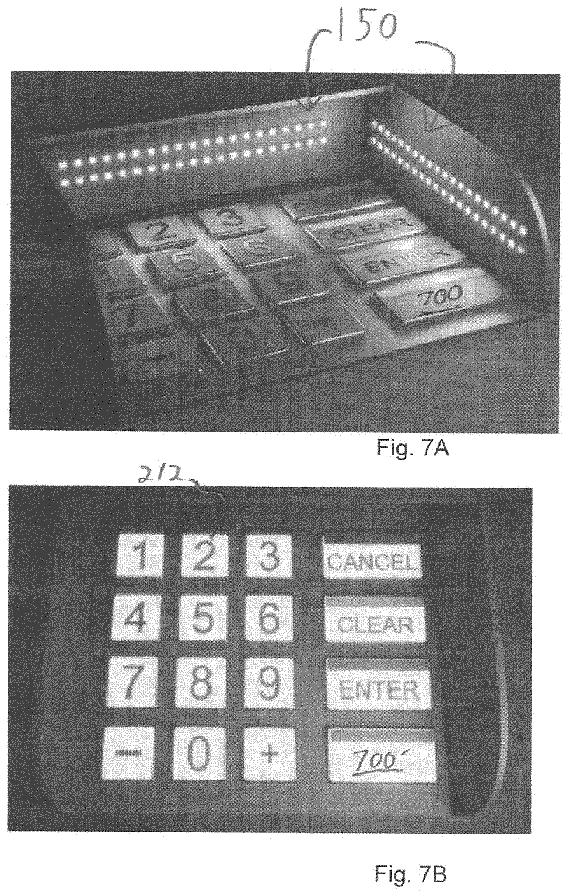

FIGS. 7A and 7B show Automated Teller Machine (ATM) usage of the devices of FIGS. 2A and 2B. FIG. 7a shows external irradiation of a keypad 700 from side-radiating light sources 150. FIG. 7B shows a backlit arrangement where the keypad 700′ is formed from the light conduction medium 212. As with the elevator buttons of FIG. 5, ATM buttons are prone to successive pressing (touch) over very short intervals by a number of patrons. The same holds true for the myriad of keypads frequently employed for monetary and other transactions. Retail point-of-sale (POS) stations frequently employ a keypad for credit/debit card transactions. Scanner guns are also used, often by self-checkout stations for use by successive customers. Alphanumeric keyboards often incur usage from multiple users, even in private settings.

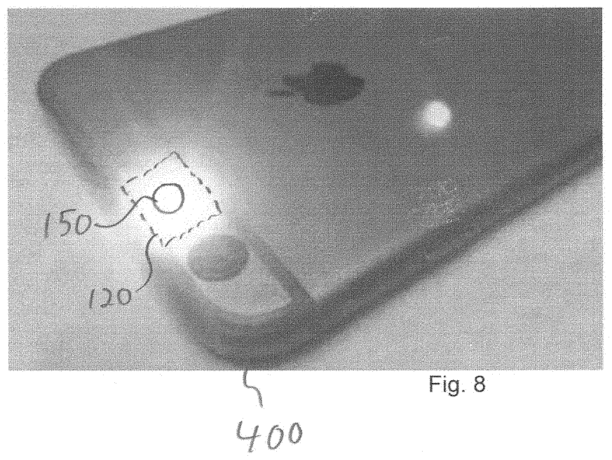

FIG. 8 shows the irradiation device embedded in a personal device for usage with a flashlight application of the personal device 400 as in FIG. 4. Personal devices often have a capability for a utility lighting with an on-board LED. Configurations herein incorporate the light source 150 with the on-board LED and embed the irradiation device 120 in the phone itself.

A myriad of high-touch Surfaces, Apparatuses, and Equipment (SAE) are used in the manufacture and deployment of various goods. Components molded from plastics and polymers are often employed and are amenable to molding from a substance functional as the light conduction medium for use as disclosed above. Some examples of such usages include but are not limited to: a lid and interior of a storage box; a lid and interior of a porch delivery box; an infant appliance drying rack; infant pacifiers, bottle nipples, or teething toys; elevator buttons; personal device and smartphone device cases; personal device and smartphone screens; touchscreens; IV pole user interface; light(s) from smartphone; keyboards; computer mouse; ATM (Automated Teller Machine) facilities and controls; numeric and telephone keypads; gas pump handles; door handles and knobs; push buttons; faucets; hand and bed railing; delivery boxes; cat litter boxes; cat and dog food and water dishes; utility lights on personal devices; housing backs for personal devices; toilet seats; toilet paper dispensers; toilet stall handles and railings; transport (airplane, automobile, bus, train, boat, subway) lighting; toothbrushes; table, countertop, chair surfaces; garbage cans; and water fountains.

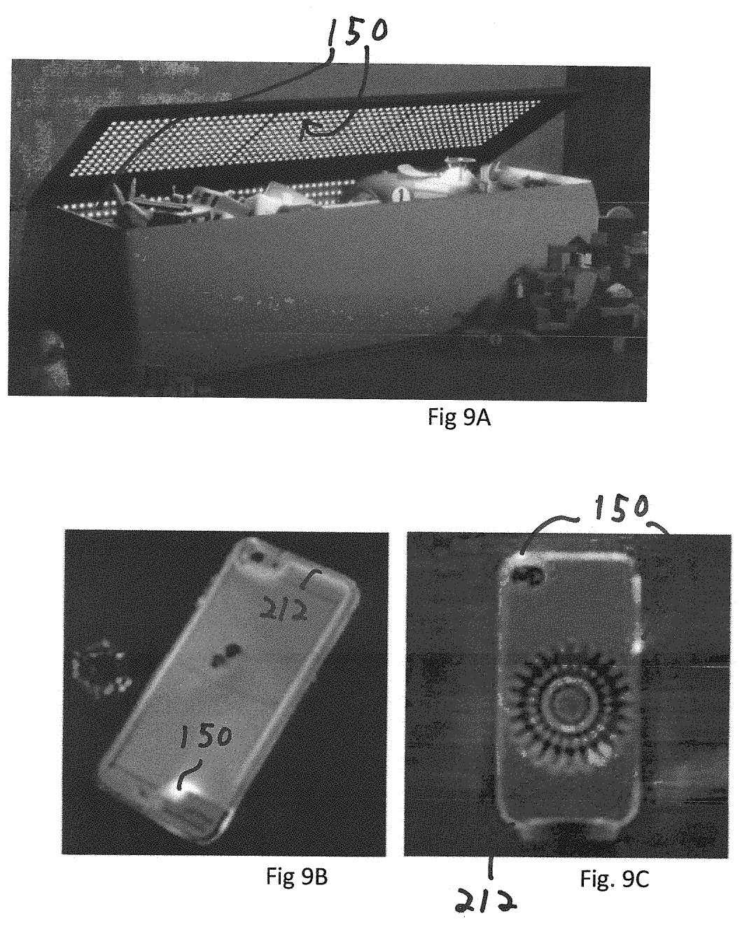

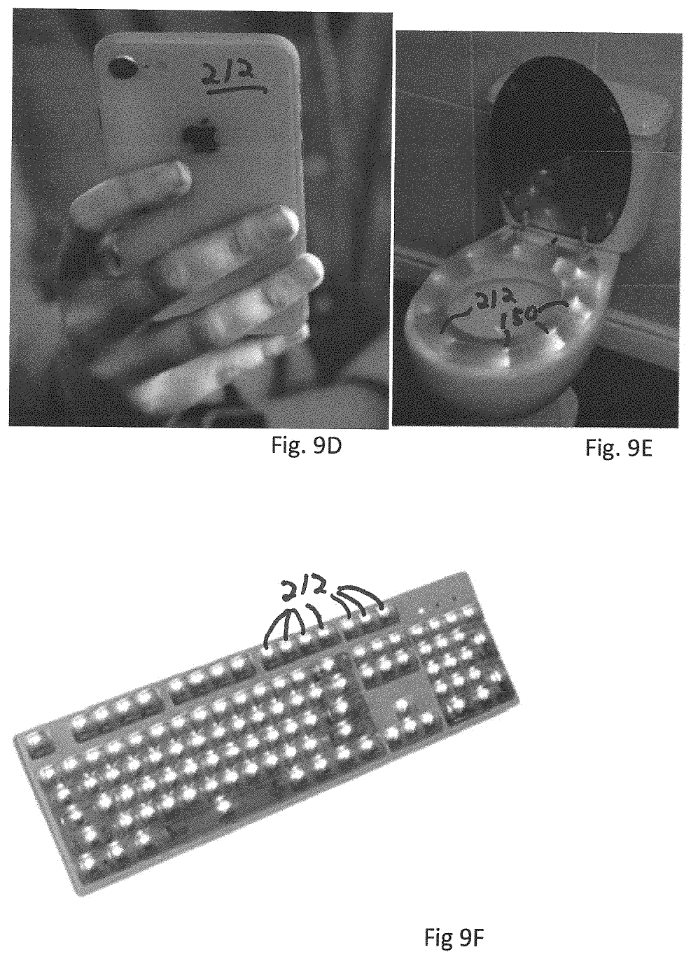

FIGS. 9A-9F show the irradiation device in a plurality of use cases and contexts. Any suitable high touch surface be fabricated from the light conduction medium. Referring to FIG. 9A, the light sources 150 are disposed along an interior of a toy or storage box for sterilization of items stored therein. Referring to FIGS. 9B, 9C and 9D, a phone case may include a plurality of light sources 150 around a case formed from the light conduction medium 212. In FIG. 9E, a toilet seat includes a contact surface of the light conduction medium 212 and includes light sources 150 dispersed in a circular array around the diameter. FIG. 9F shows a full-size keyboard including keys formed from the light conduction medium 212.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An irradiation device, comprising:
a light source disposed for irradiating a high touch surface for sterilization.

2. The device of claim 1 further comprising a light conduction medium adapted for transporting irradiated light from the light source to the high touch surface.

3. The device of claim 2 wherein the light source is appurtenant to the light conduction medium and the light conduction medium includes the high touch surface.

4. The device of claim 2 wherein the light conduction medium includes a transparent structure, the transparent structure molded to define the high touch surface.

5. The device of claim 4 wherein the transparent structure is defined by a crystalline material, the crystalline material providing non-linear pathways for irradiation of the light between the light source and the high touch surface.

6. The device of claim 1 further comprising a power supply and illumination logic, the illumination logic powered by the power supply and configured to activate the light for a duration and intensity based on a sterilization need.

7. The device of claim 6 wherein the control logic is further configured to compute the duration and intensity based on a type of material defining the high touch surface.

8. A system for sterilization of contaminated surfaces, comprising:
an antimicrobial light source having a wavelength outside a harmful spectrum; and
a light conduction medium configured to conduct light from the antimicrobial light source to a target surface for sterilization,
the antimicrobial light source having an intensity based on a duration of exposure on the target surface and a power for achieving sterilization over the duration of exposure.

9. The system of claim 8 further comprising control logic, the antimicrobial light source responsive to the control logic for:
identifying the target surface for sterilization;
determining a material of which the target surface is formed; and
computing the duration of exposure and the intensity for achieving sterilization of the target surface.

10. The system of claim 8 wherein the light conduction medium is a transparent structure appurtenant to a high touch surface associated with touch based manual control.

11. The system of claim 10 wherein the light conduction medium irradiates the target surface from a distance via an atmospheric medium.

12. The system of claim 10 wherein the light conduction medium irradiates the target surface via refraction through the transparent structure.

13. The system of claim 8 further comprising a power source for providing power to the antimicrobial light source including at least one of a DC-powered battery and an AC power source.

14. A system for sterilization of contaminated surfaces, comprising a light conduction medium formed from a material selected from the group of light-transmissive materials consisting of polymethyl methacrylate (PMMA), silica/quartz, thermoplastic polyurethane (TPU), flexible acrylic, transparent polyvinyl chloride (PVC), UV-inhibitor-free transparent PVC and solar cell material.

15. A method for forming a high touch surface, comprising:
deploying a light conduction medium having a high touch surface and adapted for transporting light from an antimicrobial light source to a high touch surface.
